(19) United States
(12) Patent Application Publication
(10) Pub. No.: US 2006/0047265 A1
(43) Pub. Date: Mar. 2, 2006

(54) Multi-exchange catheter guide member with improved seal

(75) Inventors: Kevin Boyle, Renmore (IE); David Quinn, Salthill (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/925,478

(22) Filed: Aug. 25, 2004

(51) Int. Cl. A61M 25/01

(52) U.S. Cl. 604/528

(57) ABSTRACT

A catheter and a guidewire exchange system includes a catheter and a guide member. The catheter includes a lumen extending through the shaft and sized to receive the guidewire, and a longitudinal guideway enabling transverse access from the shaft exterior surface to the lumen. The guide member includes a housing, a catheter passageway extending through the housing and adapted to slidably receive the catheter, a guidewire passageway extending from one end of the housing into the catheter passageway and including a tube adapted to merge the guidewire transversely through the guideway and into the first lumen and a rigid nose cone attached to the housing distal end and having an aperture extending therethrough that is continuous with the catheter passageway and is adapted to slidably receive the catheter.

### TECHNICAL FIELD

The present invention generally relates to medical catheters and medical apparatuses involving medical catheters. The present invention more particularly relates to Multi-Exchange catheters with improved guide members.

### BACKGROUND

Cardiovascular disease, including atherosclerosis, is a leading cause of death in the U.S. The medical community has developed a number of methods and devices for treating coronary heart disease, some of which are specifically designed to treat the complications resulting from atherosclerosis and other forms of coronary arterial narrowing.

One method for treating atherosclerosis and other forms of coronary narrowing is percutaneous transluminal coronary angioplasty, commonly referred to as “angioplasty” or “PTCA.” The objective in angioplasty is to enlarge the lumen of the affected coronary artery by radial hydraulic expansion. The procedure is accomplished by inflating a balloon of a balloon catheter within the narrowed lumen of coronary artery.

In addition to PTCA, catheters are used for delivery of stents or grafts, therapeutic drugs (such as anti-vaso-occlusion agents or tumor treatment drugs) and radiopaque agents for radiographic viewing. Other uses for such catheters are well known in the art.

The anatomy of coronary arteries varies widely from patient to patient. Often a patient's coronary arteries are irregularly shaped, highly tortuous and very narrow. The tortuous configuration of the arteries may present difficulties to the physician in proper placement of a guidewire, and advancement of a catheter to a treatment site. A highly tortuous coronary anatomy typically will present considerable resistance to advancement of the catheter over the guidewire.

Therefore, it is important for a catheter to be highly flexible. However, it is also important for a catheter shaft to be stiff enough to push the catheter into the vessel in a controlled manner from a position far away from the distalmost point of the catheter.

Catheters for PTCA and other procedures may include a proximal shaft, a transition section and a distal shaft having a flexible distal tip. In particular, the catheters have a proximal shaft, which is generally rigid for increased pushability and a more flexible distal shaft with a flexible distal tip for curving around particularly tortuous vessels. The proximal shaft may be made stiff by the insertion of a thin biocompatible tube, such as a stainless steel hypotube, into a lumen formed within the proximal shaft. The transition section is the portion of the catheter between the stiffer proximal shaft and the more flexible distal shaft, which provides a transition in flexibility between the two portions.

With some types of catheter construction, when an increase in resistance occurs during a procedure there is a tendency for portions of the catheter to collapse, buckle axially or kink, particularly in an area where flexibility of the catheter shaft shifts dramatically. Consequently, the transition section is often an area where the flexibility of the catheter gradually transitions between the stiff proximal shaft and the flexible distal shaft. It is known in the art to create a more gradual flexibility transition by spiral cutting a distal end of the hypotubing used to create stiffness in the proximal shaft. Typically, the spiral cut is longitudinally spaced farther apart at the hypotube proximal end creating an area of flexibility, and longitudinally spaced closer together at the hypotube distal end creating an area of even greater flexibility.

In a typical PTCA procedure, it may be necessary to perform multiple dilatations, for example, using various sized balloons. In order to accomplish the multiple dilatations, the original catheter must be removed and a second catheter tracked to the treatment site. When catheter exchange is desired, it is advantageous to leave the guidewire in place while the first catheter is removed to properly track the second catheter.

Two types of catheters commonly used in angioplasty procedures are referred to as over-the-wire (OTW) catheters and rapid exchange (RX) catheters. A third type of catheter with preferred features of both OTW and RX catheters, which is sold under the trademarks MULTI-EXCHANGE, ZIPPER MX, ZIPPER, MX and/or MXII, is discussed below. An OTW catheter's guidewire lumen runs the entire length of the catheter and may be positioned next to, or enveloped within, an inflation shaft. Thus, the entire length of an OTW catheter is tracked over a guidewire during a PTCA procedure. A RX catheter, on the other hand, has a guidewire lumen that extends within only the distalmost portion of the catheter. Thus, during a PTCA procedure only the distalmost portion of a RX catheter is tracked over a guidewire.

If a catheter exchange is required while using a standard OTW catheter, the user must add an extension wire onto the proximal end of the guidewire to maintain control of the guidewire, slide the catheter off of the extended guidewire, slide the new catheter onto the guidewire and track back into position. Multiple operators are required to hold the extended guidewire in place while the original catheter is exchanged in order to maintain its sterility.

A RX catheter avoids the need for multiple operators when exchanging the catheter. With a rapid exchange catheter, the guidewire runs along the exterior of the catheter for all but the distalmost portion of the catheter. As such, the guidewire can be held in place without an extension when the catheter is removed from the body. However, one problem associated with RX catheters is the guidewire, and most of the catheter, must be removed from the body in order to exchange guidewires. Essentially the procedure must then start anew because both the guidewire and the catheter must be retracked to the treatment site. An OTW catheter, with the guidewire lumen extending the entire length of the catheter, allows for simple guidewire exchange.

A balloon catheter capable of both fast and simple guidewire and catheter exchange is particularly advantageous. A catheter designed to address this need is sold by Medtronic Vascular, Inc. of Santa Rosa, Calif. under the trademarks MULTI-EXCHANGE, ZIPPER MX, ZIPPER, MX and/or MXII (hereinafter referred to as the “MX catheter”). An MX catheter is disclosed in U.S. Pat. No. 4,988,356 to Crittenden et al.; co-pending U.S. patent application Ser. No. 10/116,234, filed Apr. 4, 2002; co-pending U.S. patent application Ser. No. 10/251,578, filed Sep. 18, 2002; co-pending U.S. patent application Ser. No. 10/251,477, filed Sep. 20, 2002; co-pending U.S. patent application Ser. No. 10/722,191, filed Nov. 24, 2003; and co-pending U.S. patent application Ser. No. 10/720,535, filed Nov. 24, 2003, all of which are incorporated by reference in their entirety herein.

The MX catheter includes a catheter shaft having a guidewire lumen positioned side-by-side with an inflation lumen. The MX catheter also includes a longitudinal cut that extends along the catheter shaft and that extends radially from the guidewire lumen to an exterior surface of a catheter shaft. A guide member through which the shaft is slidably coupled cooperates with the longitudinal cut such that a guidewire may extend transversely into or out of the guidwire lumen at any location along the longitudinal cut's length. By moving the shaft with respect to the guide member, the effective over-the-wire length of the MX catheter is adjustable.

The guidewire is threaded into a guidewire lumen opening at the distal end of the catheter and out through the guide member. The guidewire lumen envelopes the guidewire as the catheter is advanced into the patient's vasculature while the guide member and guidewire are held stationary. Furthermore, the indwelling catheter may be removed by withdrawing the catheter from the patient while holding the proximal end of the guidewire and the guide member in a fixed position. When the catheter has been withdrawn to the point where the distal end of the cut has reached the guide member, the distal portion of the catheter over the guidewire is of a sufficiently short length that the catheter may be drawn over the proximal end of the guidewire without releasing control of the guidewire or disturbing its position within the patient.

While MX catheters provide many advantages over RX and OTW catheters, both RX and MX catheters need to be sealed effectively at the hemostasis valve. OTW catheters are readily sealed at the valve since the guidewire is within the catheter shaft which extends through the valve. RX and MX catheters have a catheter shaft and guidewire separated proximal to the hemostasis valve and thus an effective valve seal must take into consideration the catheter and guidewire separation for an RX catheter and with the guide member in the case of the MX catheter. For example, in a typical dye injection, the physician may pull a slight negative pressure to ensure no air bubbles are within the system prior to injecting the dye. If the physician pulls a very heavy negative pressure, there remains a possibility that air may enter the patient through the hemostasis valve if not sealed sufficiently around the catheter, guide wire and guide member of an MX catheter. Similarly, when a hemostasis valve has an active/passive gasket, if the valve is not properly closed down on an RX catheter shaft and guidewire, air may be drawn into the system when a very heavy vacuum is drawn.

Accordingly, it is desirable to provide an apparatus or system that improves shaft stability within the hemostasis valve and provides a secure seal for a Multi-Exchange catheter at the hemostatic valve location. It is also desirable to provide such an apparatus or system that can be implemented with a currently used catheter guide tool. In addition, it is desirable to provide such an apparatus that does not slow down guidewire insertion, contrast media injection, or other medical processes involving the catheter. Furthermore, other desirable features and characteristics of the present invention will become apparent from the subsequent detailed description and the appended claims, taken in conjunction with the accompanying drawings and the foregoing technical field and background.

### BRIEF SUMMARY

A system is provided for exchanging a catheter and guidewire in a patient. The system comprises a catheter and a guide member. The catheter comprises an elongate shaft having an exterior surface, a proximal end, and a distal end; a first lumen extending through the shaft from the shaft proximal end to the shaft distal end, and sized to receive a guidewire; and a longitudinal guideway extending distally from the shaft proximal end, and enabling transverse access from the shaft exterior surface to the first lumen. The guide member comprises a housing having a proximal end and a distal end; a catheter passageway extending through the housing from the proximal end to the distal end and adapted to slidably receive the catheter; a guidewire passageway extending from the housing proximal end into the catheter passageway, and comprising a tube adapted to merge the guidewire transversely through the guideway and into the first lumen; and a rigid nose cone attached to the housing distal end and having an aperture extending therethrough that is continuous with the catheter passageway and is adapted to slidably receive the catheter.

An apparatus is also provided for advancing and retracting a guidewire and a catheter having a lumen, an exterior surface, and a longitudinal guideway that enables transverse access from the catheter exterior surface to the lumen. The apparatus comprises a housing having a proximal end and a distal end; a catheter passageway extending through the housing from the proximal end to the distal end and adapted to slidably receive the catheter; a guidewire passageway extending from the housing proximal end into the catheter passageway, and comprising a tube adapted to merge the guidewire transversely through the guideway and into the first lumen; and a rigid nose cone attached to the housing distal end and having an aperture extending therethrough that is continuous with the catheter passageway and is adapted to slidably receive the catheter.

### BRIEF DESCRIPTION OF THE DRAWINGS

The present invention will hereinafter be described in conjunction with the following drawing figures, wherein like numerals denote like elements, and

FIG. 1 is a perspective view of a guide member with a guide wire extending through a guide member and into a catheter according to the present invention;

FIGS. 2A-D are cross sectional views of a catheter at points A-A, B-B, C-C, and D-D illustrated in FIG. 1;

FIG. 3 is a perspective cross sectional view of an oval proximal shaft;

FIG. 4 is a cross sectional view of a circular proximal shaft;

FIG. 5 is a sectional view of a guide member and its components according to the present invention;

FIG. 6 is a sectional view of the guide member equipped with a nose cone and various seals according to an embodiment of the present invention; and

FIG. 7 is a sectional view of the guide member equipped with a nose cone, an elongate wire, and various seals according to an embodiment of the present invention.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

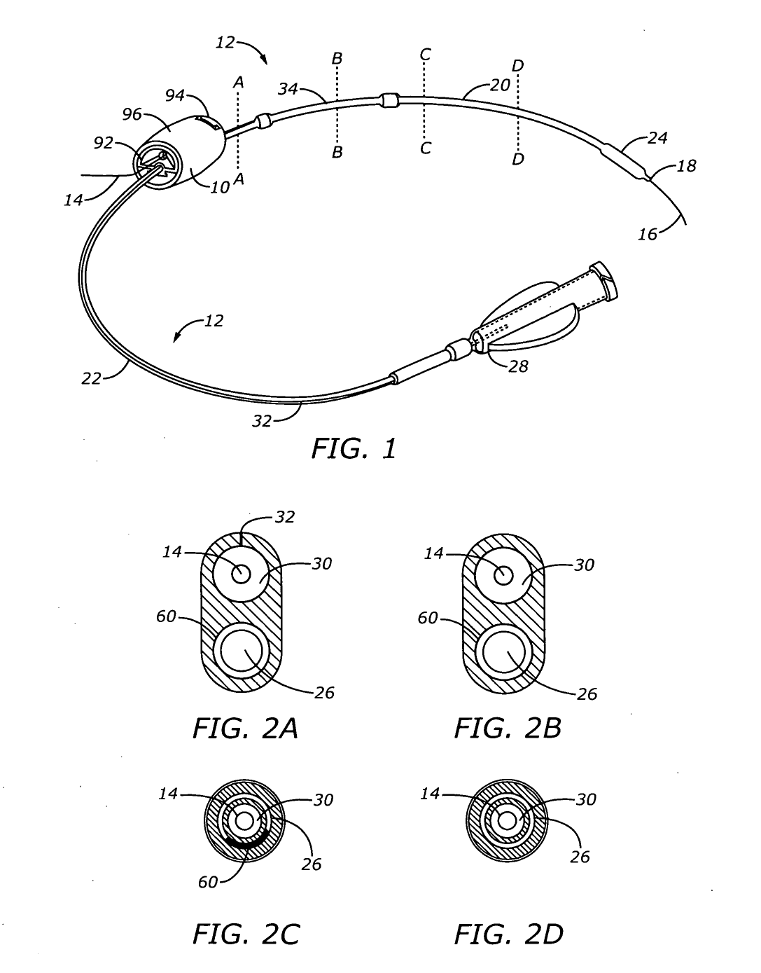

The present invention is used with an MX catheter, an exemplary embodiment of which is illustrated in FIG. 1. The catheter 12 includes an elongate, flexible, cylindrical main body having a distal shaft 20 and a proximal shaft 22. According to the present embodiment, the catheter 12 is a delivery catheter for such procedures as PTCA or stent delivery and has a balloon 24 mounted around the catheter body near the catheter distal end 18. The balloon 24 may be inflated and deflated through the catheter inflation lumen 26. The inflation lumen 26 communicates with a fitting 28 at the catheter proximal end, and extends the catheter length to terminate in communication with the balloon interior at the catheter distal end 18. The catheter 12 also includes a guidewire lumen 30 that receives the guidewire 14 and extends the entire catheter length. A longitudinal cut extends into the guidewire lumen 30 along the length of most of the proximal shaft 22 to form a guideway 32. The proximal shaft distal section 34 does not include the guideway 32. The guidewire lumen 30 and the inflation lumen 26 are coaxially arranged in the distal shaft 20 according to the present embodiment.

The present invention includes a guide member for the MX catheter 12. FIG. 1 depicts a guide member 10 according to an embodiment of the invention, with a guide wire 14 extending through the guide member 10 and into the MX catheter 12. FIGS. 2A to 2D are cross sections of the catheter 12 at points A-A, B-B, C-C, and D-D along the catheter length. The guide member 10 serves as a juncture in which the catheter 12 and guidewire 14 may be merged or separated so that the guidewire portion that extends proximal to the guide member 10 is separated from the catheter 12, and the guidewire portion that is located distal to the guide member 10 is contained and housed within the catheter, although the guidewire distal end 16 may protrude out of the catheter distal end 18.

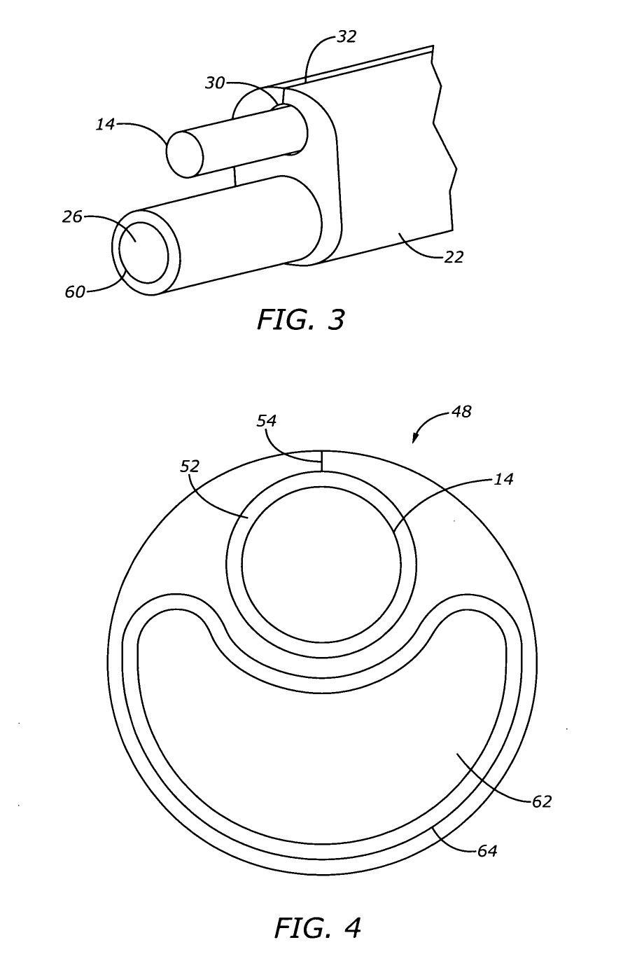

The catheter proximal shaft 22 described above can be modified to suit various needs. For example, the proximal shaft can be a tri-lumen shaft to provide passage for various drugs, fluids, wires, or other necessary compositions or equipment. Further, the proximal shaft may be oval, circular, or other suitable shape. FIG. 3 is a perspective cross sectional view of an oval proximal shaft 22 according to one embodiment of the invention, and FIG. 4 is a cross sectional view of a circular proximal shaft 48 according to another embodiment of the invention. Each of the proximal shafts 22, 48 has a respective guidewire lumen 30, 52 that is accessible through a guideway 32, 54 located along the proximal shaft length. Each of the proximal shafts 22, 48 also includes an inflation lumen 26, 62 that extends side by side with the guidewire lumen 30, 52 along the proximal shaft length. The inflation lumens 26, 62 are preferably supported by a stiffening member 60, 64 such as a hypotube. The inflation lumen 62 in the embodiment depicted in FIG. 4 is crescent shaped and the hypotube stiffening member 64 also is formed in the same shape to withstand force transmission along the catheter length. The stiffening members may further include a transition section at their respective distal sections in conjunction with a transition between the relatively stiff proximal shaft to the relatively flexible distal shaft and avoid shaft kinking at the junction therebetween. For example, the hypotube 60 may be skived at its distal end, with the skived portion extending into the distal section as depicted in FIG. 2C.

Returning to FIG. 1, the proximal shaft 22 can be formed from suitable biomedical grade materials such as polyethylene, cross-linked polyethylene, polyolefins, polyamides, blends of polyamides and polyolefins, fluoropolymers, polyesters, polyketones, polyimides, polysulphones, polyoxymethylenes, and compatibilizers based on polyolefins, including grafted polyolefins, and other comparable materials. A lubrication additive may also be used with any polymer and may include polyethylene micro-powders, fluoropolymers, silicone based oils, fluoro-ether oils, molybdenum disulphide and polyethylene oxide. Additionally, a reinforcing additive may be used such as nano-clays, graphite, carbon fibers, glass fibers, and polymeric fibers. The distal shaft 20 can be made of a suitable polyethylene or polyolefin that readily bonds to the proximal shaft 22.

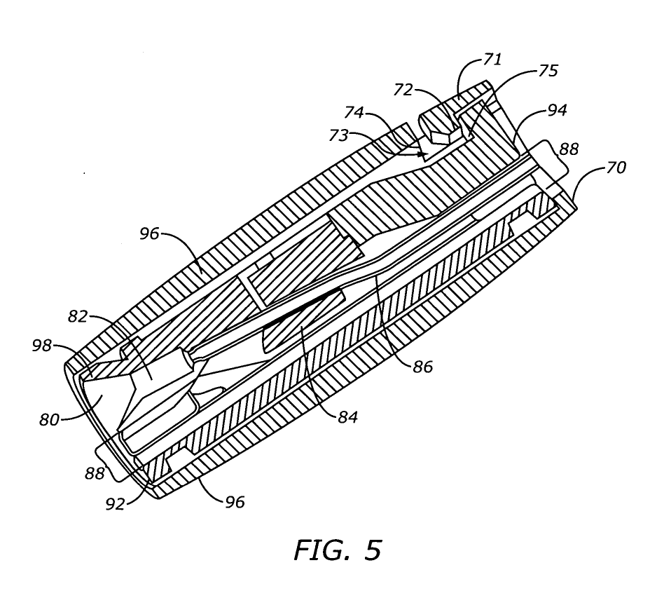

Turning now to FIG. 5, the guide member 10 and its components will be discussed according to one embodiment of the invention. The guide member 10 surrounds the proximal shaft 22 and includes proximal and distal ends 92, 94. An outer tubular member 96 freely rotates around an inner main body 98 and hence is decoupled from the inner main body 98. An inwardly extending distal annular wall 70 prevents the main body 98 from slipping out of the outer member 96. A retaining clip 71 includes a tab 72 that extends into a space 73 formed by two main body walls 74, 75. Additional tabs may be used as necessary to retain the inner main body 98 within the tubular member 96.

The guide member main body 98 includes a catheter passageway 88 extending longitudinally in a generally straight line from the guide member proximal end 92 to the guide member distal end 94. A guidewire passageway 80 extends distally from the guide member proximal end 92 through an entrance port 82 into a tube 86 and then into the catheter guidewire lumen 30, although the catheter is not depicted in FIG. 5. The catheter passageway 88 is configured to slidingly receive the proximal shaft 22, and has a cross sectional shape that approximates the proximal shaft shape, whether the proximal shaft is circular, oval, triangular, shamrock shaped, or otherwise shaped. The catheter passageway 88 enlarges in a central area to provide space for a keel 84 that is aligned with the passageway 80 and positioned to spread the catheter guideway 32 and extend into the catheter guidewire lumen 30 to enable guidewire insertion during use.

The entrance port 82 is configured to mate with a conventional wire introducer tool and is tapered to aid in loading such a tool. The tube 86 may vary in its length, although in an exemplary embodiment of the invention the tube 86 extends through the catheter guidewire lumen 30 approximately thirty-five millimeters past the guide member distal end 94. The tube 86 may be formed from a flexible material such as a polyimide, and particularly the tube region that extends through the catheter guidewire lumen 30. In one embodiment of the invention the tube region that introduces the guidewire 14 into the guidewire lumen 30 may be substantially rigid to provide the necessary support for the guidewire 14.

The guide member 10 is made of blends of polyamides and polyolefins in an exemplary embodiment of the invention. Other exemplary materials include ceramics, metals such as stainless steel, and other polymers such as polyamides and liquid crystal polymers. Lubrication additives such as polyethylene micro-powders, fluoropolymers, silicone-based oils, fluoro-ether oils, molybdenum disulphide, and polyethylene oxide may be included. Reinforcing additives such as nano-clays, graphite, carbon fibers, glass fibers, polyesters, polyketones, polyimides, polysulphones, polyoxymethylenes, polyolefins, cross-linked polyolefins may also be included, along with compatibilizers based on polyolefins, such as grafted polyolefins, ceramics, and metals.

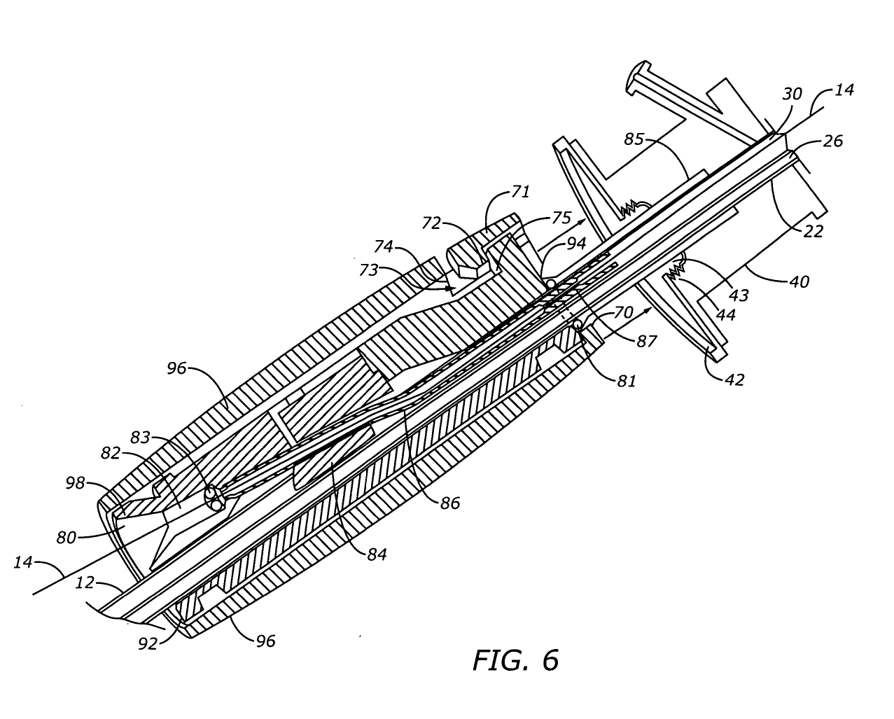

An exemplary guide member operation will now be described, although the procedures in the following description clearly set forth only one of many operations enabled by the guide member 10. The exemplary guide member operation is discussed with reference to FIG. 6, which is a sectional view of the guide member 10, a Touhy adaptor or hemostasis valve 40, and a catheter 12 extending through the catheter passageway and the hemostasis valve 40 with a guidewire 14 being directed into the guidewire lumen 30. After the guidewire 14 and a guide catheter (not shown) are inserted into a patient, the catheter 12 is inserted with a backloading operation. The guidewire 14 is inserted into the catheter distal end 18 and threaded proximally through the guidewire lumen 30 until the guidewire tube 86 captures the guidewire proximal end and directs it into the passageway 80 and then out of the guide member proximal end 92. This procedure can be accomplished with the guide member 10 adjacent the catheter guideway distal end.

As the distal shaft 20 enters the patient, the guide member 10 can be brought near the hemostasis valve 40. The guide member 10 is seated adjacent to the hemostasis valve 40 and is equipped with a rigid nose cone 85 that surrounds the distal shaft and is inserted into the hemostasis valve 40. The proximal shaft 22 is then advanced through the guide member, and the keel 84 engages the catheter guideway 32. After the catheter 12 is inserted, a gasket 42 on the hemostasis valve 40 that slidably receives the nose cone 85 is tightened to form a substantially air tight seal with the nose cone 85. Although the gasket 42 is tightened around the nose cone 85 using threaded regions 43, 44 in the embodiment depicted in FIG. 6, other known tightening methods can be used to create a substantially air tight seal around the nose cone 85.

Without the rigid nose cone 85 surrounding the distal shaft 20, the gasket 42 would typically clamp directly onto the distal shaft 20. Since the tube 86 extends in to the distal shaft 20, the tube 86 would typically be subjected to the valve clamping force. However, the rigid nose cone 85 prevents any clamping force from being exerted on the distal shaft 86 and allows the catheter 12 to be advanced and retracted as necessary without any frictional resistance from the hemostasis valve 40.

If a wire change is required, one simply withdraws the guidewire 14 from the guide member 10 as the guide member 10 is seated against the valve and as the proximal shaft 22 remains in the patient. A new guidewire is then inserted into the catheter through the passageway 80. If a catheter exchange is required, one simply holds the guidewire 14 in place and begins moving the proximal shaft 22 proximally through the guide member. Another catheter may then be backloaded onto the guidewire 14 and introduced into the patient as described above.

In order to maintain shaft stability and prevent air aspiration into the guidewire lumen 30 at the catheter proximal end, the guidewire passageway 80 is adapted to include airflow reduction components that prevent or minimize air movement through the passageway 80. In one exemplary embodiment of the invention, one such component is a seal 81 that forms an airtight seal with the nose cone 85 and the proximal shaft 22. As depicted in FIG. 6, the seal 81 may also be secured in place using the guide member distal end 94, although the seal 81 may be disposed anywhere between the nose cone 85 and the proximal shaft 22. The seal 81 prevents air that proximally enters the nose cone from entering the guidewire lumen 30 through the guideway 32. The seal 81 may be a simple o-ring structure or any other suitably sized structure that includes an inner passageway that approximates the exterior surface of the proximal shaft 22. In an exemplary embodiment, the seal 81 is formed from an elastomer that can be compressed or stretched as necessary to create an air tight seal. The seal 81 has an inner diameter or other inner passageway that approximates the proximal shaft diameter or other outer dimensions, and thereby passively provides a predetermined and consistent amount of resistance to catheter advancement and retraction. The consistent friction force allows the physician or other user to steadily advance or retract the catheter.

The passageway 80 includes at least one o-ring body 83 as another passive seal, positioned in the guidewire entrance port 82 adjacent to the tube 86. The o-ring body 83 can be formed of a flexible material, although a substantially rigid material will reduce friction with the guidewire 14. The o-ring body 83 can be positioned in any suitable location in the guidewire passageway 80 to effectively prevent or substantially minimize airflow therethrough. An exemplary location for the o-ring body 83 is the entrance port 82, although the o-ring body 83 may be disposed within the tube 86 or even the keel 84. The o-ring body 83 has an outer diameter that can approximate the inner diameter of the guidewire passageway area in which the o-ring body is positioned. The o-ring body 83 also has an inner diameter that approximates the guidewire diameter in order to provide a substantially airtight seal with the guidewire 14.

In another embodiment of the invention, the tube 86 has a substantially uniform inner diameter, but includes a fixed reduced inner diameter region 87 that prevents air movement therethrough. The term “fixed” in this sense means that the reduced inner diameter region 87 is a passive, unchanging airflow reduction body. The reduced inner diameter region 87 has a smaller inner diameter than the rest of the tube 86, or at least a smaller inner diameter than the reduced inner diameter region's immediate or nearby vicinity, and consequently substantially reduces the amount of air that flows through the tube 86 without impeding guidewire movement. The reduced inner diameter region 87 is formed distally with respect to the keel 84, and consequently is disposed inside the guidewire lumen 30 during use in an exemplary embodiment. However, the reduced inner diameter region 87 may be formed elsewhere within the passageway, and is depicted in FIG. 6 to be partially distal to the guide member 10, with the tube 86 extending beyond the guide member distal end 94 within the guidewire lumen 30.

In an exemplary embodiment depicted in FIG. 6, the polyimide or other tube material is simply manufactured to have a discrete region that has a smaller inner diameter than the rest of the tube 86, or at least a smaller diameter than that of the discrete region's immediate or nearby vicinity. However, the reduced inner diameter region 87 may be formed by slightly constricting the tube 86 using an annular body such as a bracket, a clamp, a sleeve, or other device that surrounds the tube outer surface. Alternatively, the annular body may also be attached to the tube interior surface. In another exemplary embodiment, a bracket interrupts the continuity of the polyimide or other tube material, and is manufactured in-line with the tube 86. In such an embodiment, the tube 86 is joined to the bracket by applying heat, an adhesive, or any other suitable joining tool or composition.

One reason that the reduced inner diameter region 87 is highly effective at restricting air passage through the tube 86 is the seal uniformity across the region 87. Aspiration prevention qualities are superior if a full seal entirely surrounds the tube 86. Aspiration prevention also is found to be positively related to the longitudinal length of the small diameter region 87. Consequently, doubling the small diameter region length has the effect of approximately doubling the resistance to air aspiration.

Although each of the above airflow reduction components are discussed as separate embodiments of the invention, they may be used in combination as depicted in FIG. 6. Further, using one or both of the seal 83 and the small diameter region 87 as necessary can provide the advantage of controlling blood backbleeding during a contrast medium injection process. As mentioned earlier, in one known injection process blood must be drawn into a syringe that includes the contrast media in order to remove any air bubbles that may exist in the guidewire lumen 30, and regulating blood flow into the syringe is typically performed by adjusting the hemostasis valve 40. However, hemostasis valve adjustments affect the ability for the catheter to advance or retract since the hemostasis valve is disposed about the catheter periphery, and it is problematic for the physician or other user to find the optimal hemostasis valve tightness that allows the catheter to advance or retract relatively freely, and also allows a particular amount of blood flow toward and into the syringe. The presence of the small diameter region 87, alone or together with the seal 83, enables regulated backbleed proximal to the hemostasis valve 40, thereby removing any air bubbles that may exist in the guidewire lumen 30 before injecting the contrast media with a syringe. If necessary, the backbleed may form a pool that fills a predetermined guidewire passageway portion. The pool also serves as an airflow reduction component if the guidewire is advanced or replaced with a new guidewire.

In an embodiment similar to the embodiment illustrated in FIG. 6, air aspiration can also be prevented between the tube 86 and the guidewire lumen 30 by providing the tube with an increased outer diameter region. The increased outer diameter region is a passive, unchanging airflow reduction body. The increased outer diameter region has a larger diameter than the rest of the tube 86, or at least a larger diameter than the region's immediate or nearby vicinity, and consequently substantially reduces the amount of air that flows between the tube 86 and the guidewire lumen 30 without impeding guidewire movement or preventing the catheter 12 from freely advancing or retracting. The increased outer diameter region is formed distally with respect to the keel 84, and consequently is disposed inside the guidewire lumen 30 during use in an exemplary embodiment. Similar to the reduced inner diameter depicted in FIG. 6, the polyimide or other tube material can be simply manufactured to have a discrete region that has a larger outer diameter than the rest of the tube 86, or at least a larger outer diameter than that of the discrete region's immediate or nearby vicinity. However, the increased outer diameter region may be simply attached to the tube exterior surface. In another exemplary embodiment, a bracket with a larger outer diameter than that of the tube 86 interrupts the continuity of the polyimide or other tube material, and is manufactured in-line with the tube 86. In such an embodiment, the tube 86 is joined to the bracket by applying heat, an adhesive, or any other suitable joining tool or composition.

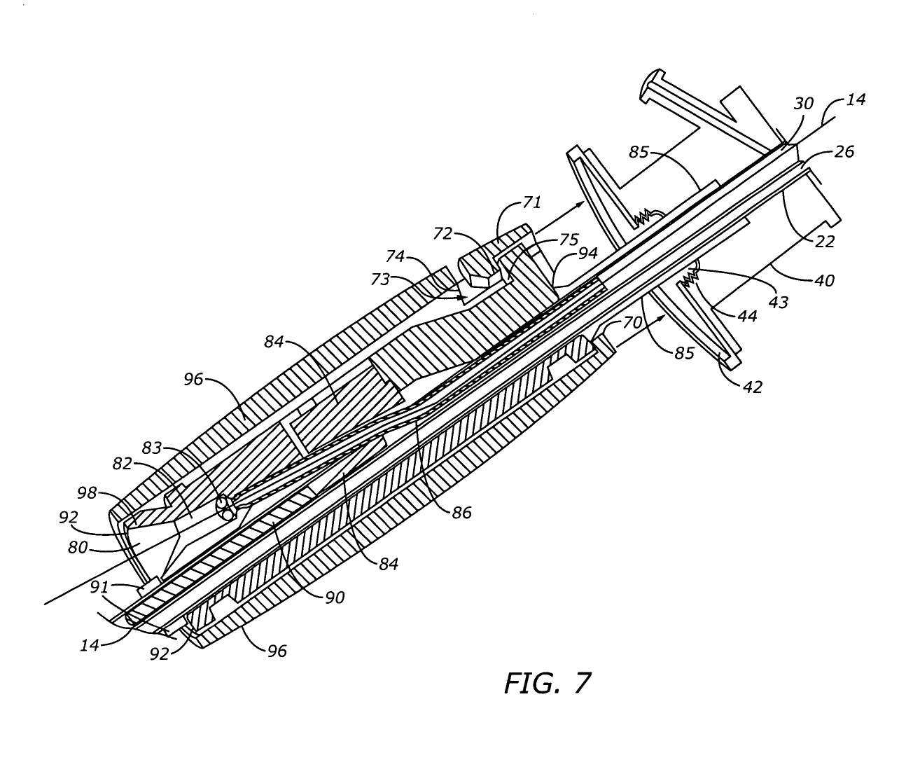

In another exemplary embodiment depicted in FIG. 7, air aspiration is further prevented by positioning a wire 90 in the guidewire lumen 30 proximal to the keel 84 as another passive seal. The wire 90 is attached to the keel 84 and plugs the guidewire passageway 80 within the keel 84 and below the tube 86. In this manner, the wire 90 and the seal 83 substantially close any potential air aspiration pathways proximal to the point where the guidewire 14 enters the guidewire lumen 30. The wire 90 is attached to the keel 84 using any suitable material or process. An exemplary wire 90 includes a lubricious material to enable the catheter 12 to freely advance or retract without substantial friction from the wire 90.

In another exemplary embodiment of the invention, a gasket 91 surrounds the catheter 12 at the guide member proximal end 92, and preferably further surrounds the lubricious wire 90 as another passive seal. The gasket can be an annular body, but has an inner passageway that approximates the exterior surface of the proximal shaft 22. The gasket 91 seals any gaps between the catheter 12 and the guide member 10 at the catheter passageway proximal end, and also ensures that the proximal shaft's longitudinal cut forming the guideway 32 into the guidewire lumen 30 is closed.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A catheter and guidewire exchange system, comprising:
a catheter, comprising:
an elongate shaft having an exterior surface, a proximal end, and a distal end,
a first lumen extending through the shaft from the shaft proximal end to the shaft distal end, and sized to receive a guidewire, and
a longitudinal guideway extending distally from the shaft proximal end, and enabling transverse access from the shaft exterior surface to the first lumen; and
a guide member, comprising:
a housing having a proximal end and a distal end,
a catheter passageway extending through the housing from the proximal end to the distal end and adapted to slidably receive the catheter,
a guidewire passageway extending from the housing proximal end into the catheter passageway, and comprising a tube adapted to merge the guidewire transversely through the guideway and into the first lumen, and
a rigid nose cone attached to the housing distal end and having an aperture extending therethrough that is continuous with the catheter passageway and is adapted to slidably receive the catheter.

2. The system according to claim 1, further comprising a seal that is positioned in the guidewire passageway and impedes airflow therethrough.

3. The system according to claim 2, wherein the seal comprises a gasket that provides a substantially airtight seal with the guidewire.

4. The system according to claim 3, wherein the guidewire passageway further comprises a guidewire entrance port that is positioned proximal to the tube, and the gasket is secured within the guidewire entrance port.

5. The system according to claim 3, wherein the gasket comprises at least one o-ring body having a central opening for slidably receiving the guidewire.

6. The system according to claim 2, wherein the seal is an integral part of the tube.

7. The system according to claim 6, wherein the seal comprises a fixed reduced diameter tube region.

8. The system according to claim 7, wherein the seal comprises an annular device secured to the tube and creating the fixed reduced diameter tube region.

9. The system according to claim 8, wherein the annular device surrounds the tube.

10. The system according to claim 8, wherein the annular device is secured to the tube interior.

11. The system according to claim 1, further comprising a seal that is positioned in the catheter passageway and impedes airflow therethrough.

12. The system according to claim 11, wherein the seal comprises a gasket that provides a substantially airtight seal with the shaft exterior surface.

13. The system according to claim 12, wherein the gasket is positioned within the nose cone pathway.

14. The system according to claim 13, wherein the gasket is further positioned adjacent to the guide member distal end.

15. The system according to claim 12, wherein the gasket is positioned adjacent to the guide member proximal end.

16. The system according to claim 11, wherein the seal comprises an elongate wire positioned inside the first lumen.

17. The system according to claim 16, wherein the guide member further comprises a keel disposed in line with the tube and having an aperture extending therethrough to slidably receive the guidewire, the keel being adapted to spread the longitudinal guideway and thereby enable transverse access from the shaft exterior surface to the first lumen.

18. The system according to claim 17, wherein at least a portion of the elongate wire is positioned proximal to the keel.

19. The system according to claim 18, wherein the elongate wire impedes airflow through the keel aperture.

20. The system according to claim 1, wherein the nose cone is sufficiently rigid to receive a clamping force from a hemostasis adaptor gasket and shield the catheter from the clamping force.

21. An apparatus for advancing and retracting a guidewire and a catheter having a lumen, an exterior surface, and a longitudinal guideway that enables transverse access from the catheter exterior surface to the lumen, the apparatus comprising:
a housing having a proximal end and a distal end,
a catheter passageway extending through the housing from the proximal end to the distal end and adapted to slidably receive the catheter,
a guidewire passageway extending from the housing proximal end into the catheter passageway, and comprising a tube adapted to merge the guidewire transversely through the guideway and into the first lumen, and
a rigid nose cone attached to the housing distal end and having an aperture extending therethrough that is continuous with the catheter passageway and is adapted to slidably receive the catheter.

22. The apparatus according to claim 21, further comprising a seal that is positioned in the guidewire passageway and impedes airflow therethrough.

23. The apparatus according to claim 22, wherein the seal comprises a gasket that provides a substantially airtight seal with the guidewire.

24. The apparatus according to claim 23, wherein the guidewire passageway further comprises a guidewire entrance port that is positioned proximal to the tube, and the gasket is secured within the guidewire entrance port.

25. The apparatus according to claim 23, wherein the gasket comprises at least one o-ring body having a central opening for slidably receiving the guidewire.

26. The apparatus according to claim 22, wherein the seal is an integral part of the tube.

27. The apparatus according to claim 26, wherein the seal comprises a fixed reduced diameter tube region.

28. The apparatus according to claim 27, wherein the seal comprises an annular device secured to the tube and creating the fixed reduced diameter tube region.

29. The apparatus according to claim 28, wherein the annular device surrounds the tube.

30. The apparatus according to claim 28, wherein the annular device is secured to the tube interior.

31. The apparatus according to claim 21, further comprising a seal that is positioned in the catheter passageway and impedes airflow therethrough.

32. The apparatus according to claim 31, wherein the seal comprises a gasket that provides a substantially airtight seal with the shaft exterior surface.

33. The apparatus according to claim 32, wherein the gasket is positioned within the nose cone pathway.

34. The apparatus according to claim 33, wherein the gasket is further positioned adjacent to the guide member distal end.

35. The apparatus according to claim 32, wherein the gasket is positioned adjacent to the guide member proximal end.

36. The apparatus according to claim 31, wherein the seal comprises an elongate wire positioned inside the first lumen.

37. The apparatus according to claim 36, wherein the guide member further comprises a keel disposed in line with the tube and having an aperture extending therethrough to slidably receive the guidewire, the keel being adapted to spread the longitudinal guideway and thereby enable transverse access from the shaft exterior surface to the first lumen.

38. The apparatus according to claim 37, wherein at least a portion of the elongate wire is positioned proximal to the keel.

39. The apparatus according to claim 38, wherein the elongate wire impedes airflow through the keel aperture.

40. The apparatus according to claim 1, wherein the nose cone is sufficiently rigid to receive a clamping force from a hemostasis adaptor gasket and shield the catheter from the clamping force.
